(19) United States
(12) Patent Application Publication
(10) Pub. No.: US 2008/0095847 A1
(43) Pub. Date: Apr. 24, 2008

(54) STIMULUS-RELEASE CARRIER, METHODS OF MANUFACTURE AND METHODS OF TREATMENT

(76) Inventors: Thierry Glauser, Redwood City, CA (US); Dariush Davalian, San Jose, CA (US); Florian Niklas Ludwig, Mountain View, CA (US); Mikael Trollsas, San Jose, CA (US)

Correspondence Address: BLAKELY SOKOLOFF TAYLOR & ZAFMAN, 1279 OAKMEAD PARKWAY, SUNNYVALE, CA, 94085-4040, US

(21) Appl. No.: 11/550,740

(22) Filed: Oct. 18, 2006

(51) Int. Cl. A61K 9/14

(52) U.S. Cl. 424/484; 424/485

(57) ABSTRACT

Compositions, methods of fabrication and methods of treatment for the controlled release of a therapeutic substance to a treatment region are disclosed herein. In some embodiments, block copolymer-based release platforms (modified or unmodified) can be used to deliver a therapeutic substance to an inflamed site in, for example, the coronary tree or the kidney glomeri. The platforms can be carriers of at least one therapeutic substance. An external “trigger”, or stimulus, such as radiation, ultrasound, temperature, a magnetic field, a change in pH, a change in ionic strength or release of an enzyme, can be used to destabilize the platform in order to release its payload in a controlled manner once at a treatment site. Delivery devices can include a syringe, an infusion catheter, a porous balloon catheter, a double balloon catheter and the like.

### FIELD OF INVENTION

Interventional cardiology.

### BACKGROUND OF INVENTION

Controlled delivery systems are systems which are designed to release a treatment agent in a controlled or sustained manner over a period of time in a patient. Such systems can include at least one polymer as a component. Examples of systems include implantable medical devices and formulations. Controlled drug delivery applications include both sustained delivery, i.e., over days, weeks, months or years, and targeted delivery, e.g., to a tumor or a diseased blood vessel, on a one-time or sustained basis. Controlled delivery systems are generally diffusion-based release systems applicable to the release of treatment agents intended for systemic circulation or for a localized site. “Diffusion” refers to form of passive transport which requires no net energy expenditure.

In some applications, the system can be an implantable medical device. In diffusion-based macromolecular release systems, the diffusion path length changes as the treatment agent leaves the device, resulting in a diminishing flux for a device such as a slab or wafer-type device. In some experimental devices, this problem can be addressed by altering the device geometry. An alternative approach is to employ relatively low treatment agent loadings and control release via degradation of the polymer using surface eroding polymers.

In some applications, the system can be a formulation. Microspheres are a clinically successful adaptation of the concept of treatment agent dispersed in a matrix in an injectable format formulation. Formation of diffusion-based microspherical treatment agent-polymer particles with dimensions typically around 1 μm to 10 μm can be accomplished by a variety of methods. In one example, treatment agent in fine powder form (approximately<0.01 μm) can be dissolved in a volatile solvent such as methylene chloride. An emulsion of the treatment agent/polymer solution in an aqueous solution containing a stabilizer is then formed, which ultimately evaporates leaving microspheres. Microspheres may also be formed by spray-drying, freezing or other techniques. Although release from these microspheres may be sustained and reproducible, the release profile is not well controlled, and injectable microspheres have found greatest application for drugs with a large therapeutic window.

### SUMMARY OF INVENTION

Compositions, methods of fabrication and methods of treatment for the controlled release of a therapeutic substance to a treatment region are disclosed herein. In some embodiments, block copolymer-based release platforms (modified or unmodified) can be used to deliver a therapeutic substance to an inflamed site in, for example, the coronary tree or the kidney glomeri. The platforms can be carriers of at least one therapeutic substance. An external “trigger”, or stimulus, such as radiation, ultrasound, temperature, a magnetic field, a change in pH, a change in ionic strength or release of an enzyme, can be used to destabilize the platform in order to release its payload in a controlled manner once at a treatment site. Delivery devices can include a syringe, an infusion catheter, a porous balloon catheter, a double balloon catheter and the like.

### BRIEF DESCRIPTION OF DRAWINGS

FIG. 1A shows an embodiment of a block copolymer.

FIG. 1B shows an alternative embodiment of a block copolymer.

FIG. 1C shows a first alternative embodiment of a block copolymer.

FIG. 2 shows a blood vessel and a first embodiment of a catheter assembly to deliver a treatment agent introduced into the blood vessel.

FIG. 3 shows a blood vessel and a second embodiment of a catheter assembly to deliver a treatment agent introduced into the blood vessel.

FIG. 4 shows a blood vessel and a third embodiment of a catheter assembly to deliver a treatment agent introduced into the blood vessel.

FIG. 5 shows a blood vessel and a fourth embodiment of a catheter assembly to deliver a treatment agent introduced into the blood vessel.

FIG. 6 shows a syringe to deliver a treatment agent introduced into the blood vessel.

### DETAILED DESCRIPTION

“Arteriosclerosis” refers to the thickening and hardening of arteries. “Atherosclerosis” is a type of arteriosclerosis in which cells including smooth muscle cells and macrophages, fatty substances, cholesterol, cellular waste product, calcium and fibrin build up in the inner lining of a body vessel. If unstable or prone to rupture, the resultant build-up is commonly referred to as vulnerable plaque. It is generally believed that atherosclerosis begins with damage to the inner arterial wall resulting in a lesion. At the damaged site, substances such as lipids, platelets, cholesterol, cellular waste products and calcium deposit in the vascular tissue may accumulate, leading to plaque progression and potentially the formation of vulnerable plaque. In turn, these substances lead to recruitment of cells involved in the inflammatory cascade of the immune system, such as macrophages, which may release substances leading to plaque destabilization.

Artherosclerotic lesions are characterized by a high content of macrophages which contribute to atherosclerosis by, for example, releasing free radicals, synthesizing bioreactive lipids, synthesizing complement components, synthesizing coagulation cascade components, secreting proteases and protease inhibitors, secreting cytokines and chemokines and phagocytosis of apoptic cells. “Apoptosis” is the disintegration of cells into membrane-bound particles that are then eliminated by phagocytosis or by shedding. Macrophage apoptosis at lesioned sites of a blood vessel can result in the decrease of inflammation.

According to embodiments of the present invention, block copolymer release platforms can be used to deliver a therapeutic agent to a specific site within a body. A “block copolymer” is a block polymer in which adjacent blocks are constitutionally different, i.e., each block comprises constitutional units derived from different characteristic species of monomer or with different composition or sequence distribution of constitutional units. Block copolymers can include di-block, tri-block or multi-block copolymers. In some embodiments, the platform can include a hydrophobic region and a hydrophilic region. That is, the block copolymer can be amphiphilic. The hydrophobic region is generally of a hydrocarbon nature, while the hydrophilic region generally includes ionic or uncharged polar functional groups.

In some embodiments, the block copolymer can be formulated into a biocompatible, bioerodable carrier. Examples of constructs which can be formulated using block copolymer constituents include, but are not limited to, hydrogels, micelles, polymerosomes, microparticles, nanoparticles and coatings. In addition, the construct can include at least one therapeutic agent. In some embodiments, the therapeutic agent can have a direct therapeutic effect on a treatment site, such as a lesion in a blood vessel. For example, in some embodiments, the therapeutic agent can reduce inflammation. Examples of such therapeutic agents include, but are not limited to, corticosteroids and anti-oxidants. In some embodiments, the therapeutic agent can have an affinity for macrophages which, when taken in by macrophages, induce macrophage apoptosis. Examples of such therapeutic agents include, but are not limited to, bisphosphonates. Other therapeutic agents include, but are not limited to, anti-proliferatives and anti-fibrotics. In one embodiment, the therapeutic agent is apolipoprotein-1 (Apo A1). Apo A1, a constituent of the cholesterol carrier high density lipoprotein (HDL), is involved in reverse cholesterol transport. Its presence can stimulate the release of cholesterol from the walls of an occluded blood vessel. Alternatively, the bioactive agent may be a peptide mimicking the function of Apo A1 protein, or a “biomimetic.”

In some embodiments, the block copolymer can be formulated with at least one labile bond or a cleavable chemical moiety. The labile bond or chemical moiety can act as a receptor to an external trigger. When the labile bond or chemical moiety receives the trigger, the block copolymer construct becomes destabilized resulting in the release of a therapeutic agent. Reception of the trigger can result in an accelerated rate of therapeutic agent release relative to lack of receipt of a trigger in the same construct. In some embodiments, the acceleration can be from at least ten times the release rate upon receipt of the trigger. The trigger can be a physical stimulus or a chemical stimulus. For example, a physical stimulus can be temperature, electrical field, pressure, sound or radiation. A chemical stimulus can be, for example, an enzyme, a peroxide or a sugar which results in a change in the pH environment or ionic environment or a change in the chemical nature of the block copolymer at the treatment site. In this manner, sustained-release of a therapeutic agent can be actively controlled by the trigger.

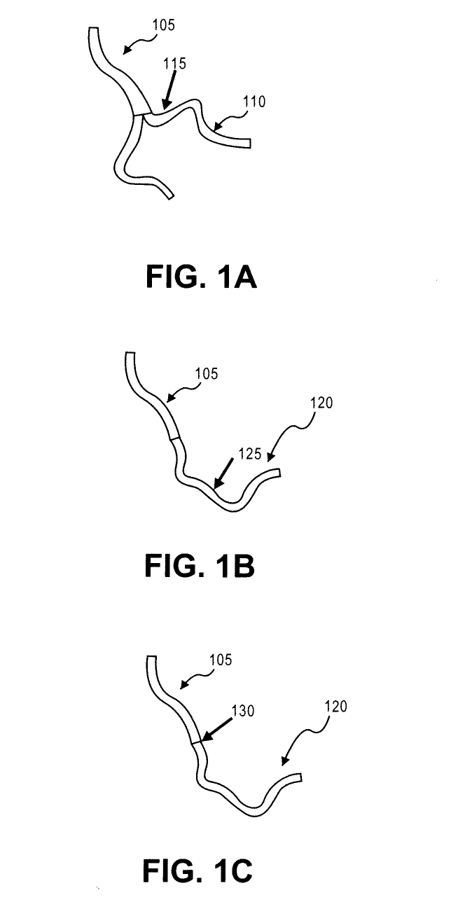

Generally, embodiments according to the present invention can follow a number of mechanisms for releasing its payload, e.g., its therapeutic agent. In one embodiment, the block copolymer can consist of a hydrophilic region 105 and a branched hydrophobic region 110 (FIG. 1A). If the block copolymer is cleaved at arrow 115, the aqueous solubility of the resulting block copolymer fragment increases, leading to an increased rate of dissociation of these resulting block copolymer fragments from the block copolymer construct, thereby destabilizing the block copolymer construct. In other embodiments, the block copolymer can consist of a shorter hydrophilic region 105 (relative to the hydrophobic region) and a longer hydrophobic region 120 (FIGS. 1B-1C). If the block copolymer is cleaved at arrow 125, an asymmetric amphiphilic block copolymer with shortened hydrophobic chain length, and therefore with increased aqueous solubility, can be realized, leading to an increased rate of dissociation of these resulting block copolymer fragments from the block copolymer construct. If the block copolymer is cleaved at arrow 130, i.e., at the junction where the hydrophobic region and the hydrophilic region are joined, the block copolymer loses its hydrophilic stealth corona which may lead to increased partitioning and retention in lipid-rich areas, e.g. in atherosclerotic plaque, and/or to precipitation or phagocytosis.

In any of the above described embodiments, the hydrophobic portion can be, but is not limited to, polylactide, polycaprolactone, polyglycolide, poly(butyl acrylate) or parylene. Similarly, the hydrophilic portion can be, but is not limited to, poly(ethylene glycol), polyvinyl alcohol), poly(vinyl pyrrolidone), poly(phosphorylcholine), hyaluronic acid, alginate or collagen.

In one embodiment, the block copolymer can be a result of a cross-linking process of homopolymers wherein two different types of polymers react to form an amphiphilic block copolymer. For example, a collagen chain may be joined with N-hydroxy succinimide ester (NHS)-functionalized polyethylene glycol to create a collagen-PEG block copolymer. Nano-constructs such as polymerosomes or micelles may be prepared from such block-copolymers.

In an alternative embodiment, the block copolymer can be a result of a crosslinking process of homopolymers wherein two different types of polymers react to form a covalent network. For example, collagen can form a cross-linked gel with N-hydroxy succinimide ester (NHS)-functionalized polyethylene glycol. Such a network can have a controlled nano-structure if prepared under controlled conditions. Such a network may be destabilized through cleavage of cross-linking chains to disintegrate the network into unconnected molecules.

### Mechanisms

In some embodiments, the trigger mechanism can be a change in pH. Some block copolymers are sensitive to the pH of their environment. Thus, when exposed to high or low pH (relative to physiological pH, or about 7 to about 7.2), the physical network of the block copolymers can be lost or modified. As a result, the construct can release its payload. Examples of copolymers sensitive to pH include, but are not limited to, polylactide-poly(ethylene glycol) (PLA-PEG), polylactide-poly(phenylene oxide) (PLA-PPO), poly(allyl amine hydrochloride)-poly(acrylic acid) (PAH-PAA), poly(dimethylaminoethyl methacrylate)-poly(methyl methacrylate) (PDMAEMA-pMMA), poly(acrylamide)-poly(methyl methacrylate) (PAAm-pMMA), poly(ethylene glycol)-poly(methyl methacrylate) (PEG-pMMA), poly(ethylene glycol)-collagen (PEG-collagen) and poly(ethylene glycol)-chitosan (PEG-chitosan). The polymer constituents of the block copolymer can be linked by a labile bond, including, but not limited to, a disulfide, an ortho-ester, an anhydride or a thio-ester. The placement of the labile bond contemplates at least the mechanisms discussed with respect to FIGS. 1A-1C.

Conversely, destabilization of the polymers and their respective constructs may be triggered or accelerated by exposure of the construct to physiological pH as compared to the pH at which the construct is stored. Thereby, the construct is stable under storage conditions but will disintegrate and release the payload after delivery to a treatment site.

### EXAMPLE

A block copolymer of PEG and pMMA linked by a degradable group such as a disulfide group can be prepared as follows. Mercaptoethanol with a protected thiol group can be coupled to an atom transfer radical polymerization (ATRP) initiator such as α-bromoisobutyryl bromide. An excess of α-bromoisobutyryl bromide can be added to a thiol-terminated PEG (mw=5 to 20 kDa) to yield a PEG macroinitiator construct containing a disulfide moiety. The PEG-containing macromolecules can be purified by recrystallization. Finally, MMA (mw=5 to 30 kDa) is polymerized using standard ATRP conditions with vitamin B in a reaction vessel to yield the diblock copolymer PEG-pMMA linked by a labile disulfide bond.

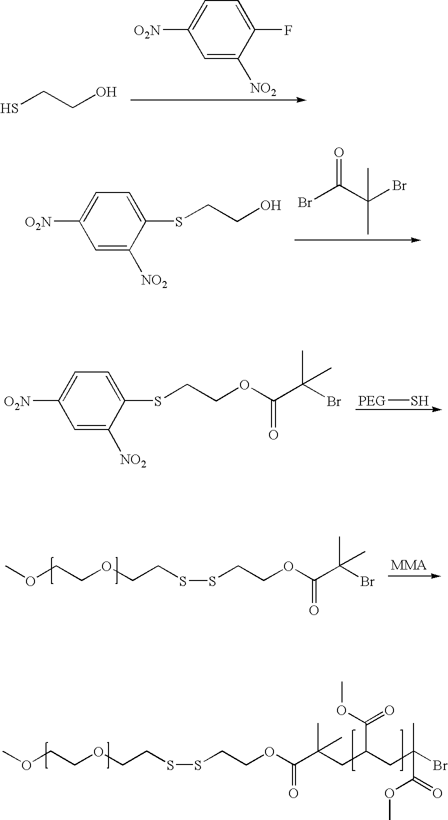

In some embodiments, the trigger mechanism can be a change in temperature. Some block copolymers are sensitive to the temperature of their environment, Thus, when temperature is increased or decreased (relative to physiological temperature, or about 37° C.), the physical structure of the block copolymer can be destroyed thereby releasing its payload. Other than those block copolymer constructs listed as pH-sensitive (which are also temperature sensitive), temperature-sensitive polymers can include, but are not limited to, PLA-co-PEG, N-isopropylacrylamide-methacrylic acid-co-octadecyl acrylate and 2-hydroxyethylmethacrylate-co-N-isopropylacrylamide. At an inflammation site, the temperature is generally greater than physiological temperature. “Inflammation” is a manifestation of the body's response to tissue damage and infection. Thus, it is anticipated that a temperature-sensitive block copolymer stimulated by a trigger such as the local environment of an inflamed site, i.e., an increase in temperature, will be disrupted thereby releasing its payload. In some embodiments, a temperature of between about 38° C. and about 60° C. will cause disruption of the block copolymer. In some embodiments, an external temperature source can be directed to the treatment site to disrupt the construct, such as microwaves or infrared radiation.

In some embodiments, a trigger mechanism can be a light source. For example, a block copolymer can be modified by adding a photolabile group. Examples of photolabile groups include, but are not limited to, triazene (—N═N—N—), diazosulfide (—N═N—S—), in addition to the following:

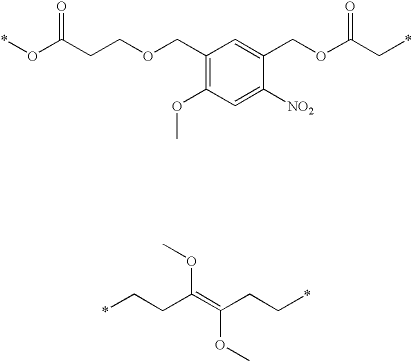

Thus, when a light source is directed to the block copolymer once it is delivered to a treatment site, the block copolymer can be disrupted releasing its payload. In some embodiments, an ultraviolet light can serve as the external trigger. Examples of wavelengths which can be effective in cleaving the photolabile group range from about 190 Å to about 11000 Å. Examples of block copolymers (diblocks, triblocks and multiblocks) polymers which can be modified at the polymer chain end or within the polymer chain with a photolabile group include but are not limited to PEG-co-PLA, PEG-co-PPO, PEG-co-PLGA and polystyrene-co-butadiene. Synthesis of such modified copolymer constructs are known by those skilled in the art.

In some embodiments, a trigger mechanism can be an enzyme. For example, a block copolymer can be modified by adding a chemical moiety susceptible to enzymatic cleavage. An “enzyme” is a protein which, in some biochemical applications, has the capability of breaking chemical bonds. An example of such a chemical moiety includes, but is not limited to, a disulfide bond. Disulfides are generally susceptible to reductase. Another example is a peptide sequence which can specifically be degraded by protease which can selectively cleave a disulfide bond. “Proteases” are enzymes that break peptide bonds between amino acids. Thus, once an enzyme is released or activated within the vicinity of the modified block copolymer, it is anticipated that the block copolymer can release its payload. In some embodiments, block copolymer cleaving enzymes are native to the treatment site, and may be found in their activated state at the treatment site. In some embodiments, the enzyme can be simultaneously delivered to a treatment site with the block copolymer. The enzyme may be delivered in a sustained release formulation. In some embodiments, the enzyme can be immobilized and made unreactive on the block copolymer until it reaches a treatment site. Once the target region is reached, e.g., the treatment site, a chemical which activates the enzyme can be delivered. Examples of block copolymers which can be subjected to enzymatic cleavage include those discussed previously.

In some embodiments, the trigger mechanism can be a chemical change in local environment. In some embodiments, the block copolymer is sensitive to its chemical environment. For example, a block copolymer can be modified by adding a chemical bond which is susceptible to a chemical stimulus (other than pH). Examples of such chemical bonds include, but are not limited to, disulfide and ester bonds. In some embodiments, such bonds are sensitive to oxidative stress. In the presence of, for example, peroxides, such bonds may be disrupted thereby disrupting the block copolymer construct. In some embodiments, the construct comprising the block copolymers is sensitive to its chemical environment, e.g. the osmotic strength of their environment. For example, if a polymerosome is prepared and stored in a hyper-osmotic environment (as compared to physiological conditions), the construct will come under osmotic stress due to influx of water when delivered into a physiological environment. In this case, a block copolymer construct can release its payload when the construct swells as a result of delivery into physiological conditions at the treatment site. Alternatively, drug release from a construct prepared at physiological osmolarity may be triggered through change of the osmotic environment local to the treatment site. For example, when the osmotic strength is decreased through delivery of a hypo-osmotic solution to the treatment site after delivery of the drug-loaded polymerosome, the polymerosome will destabilize, thereby releasing its payload.

In some embodiments, the trigger mechanism can be the application of sound or a magnetic field. For example, ethylene vinyl alcohol hydrogel can be activated by ultrasound, which can increase temperature in the polymer by weakening its physical cross-links. In some embodiments, a block copolymer can be modified to include a magnetic group, such as:

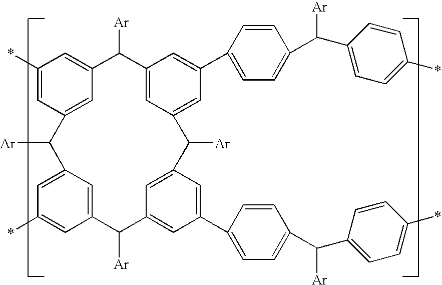

### A strong magnetic field applied to this construct can disrupt the organization of the polymer by aligning all of the dipoles.

It should be appreciated that any of the above-described embodiments may be combined in any manner. For example, an enzyme can be used to change the local environment at a treatment site (ionic change or pH change). In one embodiment, glucose oxidase can be immobilized at the surface of a pH-sensitive block copolymer construct such as pMMA-PEG by coupling it with a reactive chain end. Glucose oxidase binds to β-D-glucose (an isomer of the six carbon sugar, glucose) and aids in breaking the sugar down into its metabolites. Glucose oxidase is a dimeric protein which catalyzes the oxidation of β-D-glucose into D-glucono-1,5-lactone which then hydrolyzes to gluconic acid. Glucose oxidase requires the co-factor flavin adenine dinucleotide (FAD) in order to work as a catalyst in a redox reaction. In the glucose oxidase catalyzed redox reaction, FAD works as the initial electron acceptor and is reduced to FADH₂. FADH₂ is then oxidized by the final electron acceptor, molecular oxygen (O₂). Thereafter, O₂ is then reduced to hydrogen peroxide (H₂O₂). An increase in pH caused by the increase of hydrogen peroxide can therefore realized in the local environment around the treatment site. In some embodiments, a solution containing glucose can be co-delivered to the treatment site to increase the amount of substrate with which the glucose oxidase can bind.

In some embodiments, a trigger mechanism (such as those described above) can be used to change the chemistry of an attached group on a block copolymer construct resulting in the destabilization of the block copolymer and subsequent release of its payload. For example, in the following block copolymer backbones, an X-Y moiety can be introduced:

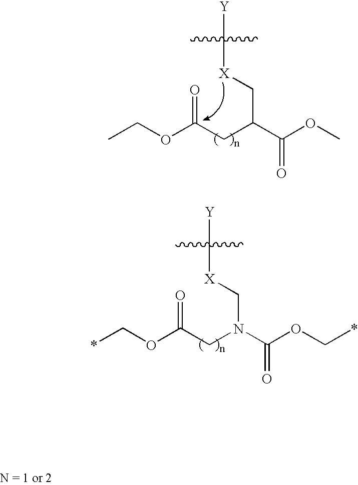

where n=1 or 2. The Y moiety is a protecting group for X that, until cleavage, is attached to X and prevents X from being a nucleophile. When Y is cleaved, X becomes an active nucleophile and attacks the carbonyl group on the block copolymer backbone creating a 5- or 6-membered ring. As a result, the block copolymer degrades and releases its payload. The following is a table of X-Y groups and a corresponding stimulus trigger:

TABLE 1

| | X-Y Group | Stimulus |
|---|---|---|
| 1 | 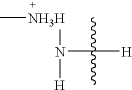 | Stable at low pH,unstable at high pH |
| 2 | 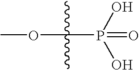 | Cleaved byphosphatase |
| 3 | 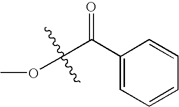 | Cleaved at high pH |
| 4 | 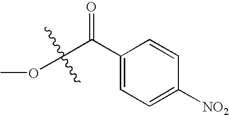 | Cleaved at higher pH |
| 5 | 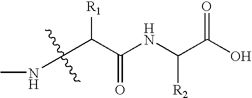 | Cleaved by protease |
| 6 | 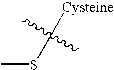 | Cleaved by reductase |
| 7 | 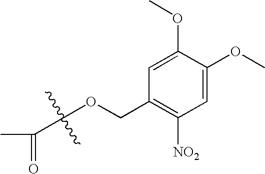 | Cleaved by radiation(light) |
| 8 | 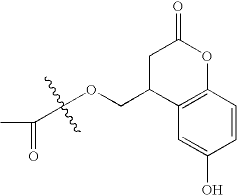 | Cleaved by radiation(light) |
| 9 | 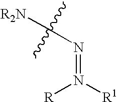 | Cleaved at low pH(R, R¹, R₂ = aromaticor alkyl chain) |

### Carriers

In some embodiments, the modified block copolymer can be formulated into a biodegradable carrier construct. Examples of biodegradable carriers include, but are not limited to, polymerosomes, micelles, particles and gels. Examples of particles include, but are not limited to, a microsphere, a nanosphere, a microrod and a nanorod.

In some embodiments, the carrier construct can provide controlled release of a therapeutic agent when activated by a trigger or stimulus. Controlled release may be beneficial when a deliberate delivery of the therapeutic agent is desirable. Because the environment in a blood vessel is subjected to constant pressure and movement of blood, a carrier construct may provide a longer duration time in which the therapeutic agent is present as the carrier degrades after being subjected to a stimulus, thereby releasing its load.

In some embodiments, the modified block copolymer can be formulated into a polymerosome. “Polymerosomes” are polymer vesicles formed from di-block or tri-block copolymers with blocks of differing solubility. Polymerosomes may be formed by methods such as film rehydration, electro-formation and double emulsion. See, e.g., Ahmed, F. et al., Self-porating polymerosomes of PEG-PLA and PEG-PCL: hydrolysis-triggered controlled release vesicles, J. Controlled Release, Vol. 96 pp. 37-57 (2004); Hammer, D. A. et al., Synthetic Cells-Self-Assembling Polymer Membranes and Bioadhesive Colloids, Annu. Rev. Mater. Res., Vol. 31 pp. 387-404 (2001). In some embodiments, a polymerosome can be a di-block copolymer including a block which is hydrophobic, e.g., poly lactic acid, polycaprolactone, n-butyl acrylate, and another block which is hydrophilic, e.g., poly(ethylene glycol), poly(acrylic acid). A polymerosome can be in a range from between about 25 nm to about 5000 nm.

In some embodiments, the modified block copolymer can be formulated into a micelle. A “micelle” is an aggregate of surfactant or polymer molecules dispersed in a liquid colloid. Micelles are often globular in shape, but other shapes are possible, including ellipsoid, cylindrical, discoid, worm-like configurations. The shape of a micelle is controlled largely by the molecular geometry of its surfactant or polymer molecules, but micelle shape also depends on conditions such as temperature or pH, and the type and concentration of any added salt. Micelles can be formed from individual block copolymer molecules, each of which contains a hydrophobic block and a hydrophilic block. The amphiphilic nature of the block copolymers enables them to self-assemble to form nanosized aggregates of various morphologies in aqueous solution such that the hydrophobic blocks form the core of the micelle, which is surrounded by the hydrophilic blocks, which form the outer shell. The inner core of the micelle creates a hydrophobic microenvironment for a non-polar therapeutic agent, while the hydrophilic shell provides a stabilizing interface between the micelle core and an aqueous medium. Examples of polymers which can be used to form micelles include, but are not limited to, polylactic acid poly ethylene glycol, polycaprolactone polyethylene oxide blocks, polyethylene oxide-β-polypropylene oxide-β-polyethylene oxide triblock copolymer and copolymers which have a polypeptide or polylactic acid core-forming block and a polyethylene oxide block. Spherical micelles are typically in a range from between about 10 nm to about 200 nm.

In some embodiments, the modified block copolymer can be formulated into a nano or micro-particle. Various methods can be employed to formulate and infuse or load the particles with a therapeutic agent, such as spray-drying and emulsion methods. For example, the particles can be prepared by a water/oil/water (W/O/W) double emulsion method. In the first phase, an aqueous phase containing treatment agent, is dispersed into the oil phase consisting of polymer dissolved in organic solvent (e.g., dichloromethane) using a high-speed homogenizer. Examples of sustained-release polymers include, but are not limited to, poly(D,L-lactide-co-glycolide) (PLGA), PLA or PLA-PEEP co-polymers, poly-ester-amide co-polymers (PEA) and polyphosphazines. The primary water-in-oil (W/O) emulsion is then dispersed in an aqueous solution containing a polymeric surfactant, e.g., poly(vinyl alcohol) (PVA), and further homogenized to produce a W/O/W emulsion. After stirring for several hours, the particles are collected by filtration. A microparticle can be in a range from about 1 μm to about 200 μm, preferably 5 μm to 50 μm. A nanoparticle can be in a range from between about 10 nm to about 1000 nm, preferably about 100 nm to about 800 nm.

### Example of Method of Treatment with Microparticles

Therapeutic agent-loaded microparticles of PEG-SS-pMMA where SS is a disulfide bond can be fabricated by spray drying or emulsion methods (such as those described above). The therapeutic agent can be, for example, bisphosphonate which induces apoptosis when phagocytized by macrophages. The microparticles can be re-dispersed in an isotonic saline solution prior to delivery to a patient to yield a particle with a hydrophilic PEG corona, i.e., a stealth particle. The solution can be injected directly to the coronary tree, or, alternatively, intravenously. The disulfide bond is sensitive to the presence of peroxides or superoxides. Therefore, the disulfide bond will degrade when it reaches a site with inflammation, the particle thereby shedding its protective corona i.e., the hydrophilic portion. These “naked”, i.e., particles without the protective hydrophilic coat-particles will be detected and phagocytized by macrophages, thereby reducing inflammation in the coronary tree. Alternatively, the de-coating of the particles may be used to retain particles at sites of lipid-rich atherosclerotic plaque through increasing the partitioning coefficient of the particles into these areas, or alternatively through mediating precipitation of the particles by shedding of the hydrophilic surface coat.

In some embodiments, the modified block copolymer can be formulated into a gel. A “gel” is an apparently solid, jelly-like material formed from a colloidal solution. By weight, gels are mostly liquid, yet they behave like solids. In some embodiments, the gel is a solution of degradable polymers. For example, the gel can be PLA in benzyl benzoate. In some embodiments, the gel is a biodegradable, viscous gel. For example, the gel can be a solution of sucrose acetate isobutyrate. In the case where the gel consists of a water-miscible organic solvent plus a polymer, a process of phase inversion occurs when the gel is introduced into the body. As the solvent diffuses out, and the water diffuses in, the polymer phase inverts, or precipitates, forming a depot of varying porosity and morphology depending on the composition. Gels can also consist of water-soluble polymers in an aqueous carrier. These can provide a faster release of therapeutic agent.

### Delivery Devices

Devices which can be used to deliver a modified block copolymer construct, include, but are not limited to, direct injection devices such as a syringe, as well as percutaneous transluminal delivery devices such as an infusion catheter, a porous balloon catheter, a double balloon catheter and the like.

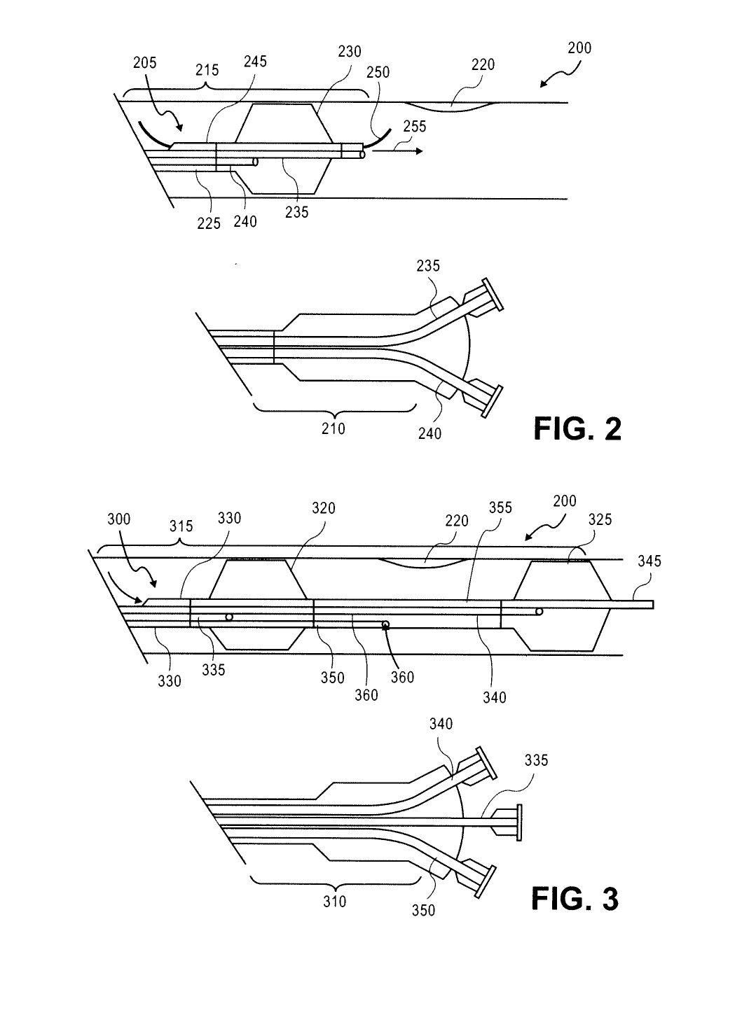

FIG. 2 shows blood vessel 200 having catheter assembly 205 disposed therein. Catheter assembly 205 includes proximal portion 210 and distal portion 215. Proximal portion 210 may be external to blood vessel 200 and to the patient. Representatively, catheter assembly 205 may be inserted through a femoral artery and through, for example, a guide catheter and with the aid of a guidewire routed to a location in the vasculature of a patient. That location may be, for example, a coronary artery. FIG. 2 shows distal portion 215 of catheter assembly 205 positioned proximal or upstream from treatment site 220.

In one embodiment, catheter assembly 205 includes primary cannula 225 having a length that extends from proximal portion 210 (e.g., located external through a patient during a procedure) to connect with a proximal end or skirt of balloon 230. Primary cannula 225 has a lumen therethrough that includes inflation cannula and delivery cannula 235. Each of inflation cannula 240 and delivery cannula 235 extends from proximal portion 210 of catheter assembly 205 to distal portion 215. Inflation cannula 240 has a distal end that terminates within balloon 230. Delivery cannula 235 extends through balloon 230.

Catheter assembly 205 also includes guidewire cannula 245 extending, in this embodiment, through balloon 230 through a distal end of catheter assembly 205. Guidewire cannula 245 has a lumen sized to accommodate guidewire 250. Catheter assembly 205 may be an over the wire (OTW) configuration where guidewire cannula 245 extends from a proximal end (external to a patient during a procedure) to a distal end of catheter assembly 205. Guidewire cannula 245 may also be used for delivery of a free treatment agent or a treatment agent encapsulated, suspended, disposed within or loaded into a biodegradable carrier when guidewire 250 is removed with catheter assembly 205 in place. In such case, separate delivery cannula (delivery cannula 235) may be unnecessary or a delivery cannula may be used to delivery one treatment agent while guidewire cannula 245 is used to delivery another treatment agent.

In another embodiment, catheter assembly 200 is a rapid exchange (RX) type catheter assembly and only a portion of catheter assembly 200 (a distal portion including balloon 230) is advanced over guidewire 250. In an RX type of catheter assembly, typically, the guidewire cannula/lumen extends from the distal end of the catheter to a proximal guidewire port spaced distally from the proximal end of the catheter assembly. The proximal guidewire port is typically spaced a substantial distance from the proximal end of the catheter assembly. FIG. 2 shows an RX type catheter assembly.

In one embodiment, catheter assembly 205 is introduced into blood vessel 200 and balloon 230 is inflated (e.g., with a suitable liquid through inflation cannula 240) to occlude the blood vessel. Following occlusion, a solution including a modified block copolymer construct can be introduced through delivery cannula 235 (arrow 255). A suitable solution of a modified block copolymer construct is about 10 to about 2000 μL in preferably an isotonic solution at physiologic pH (i.e. phosphate buffered saline). By introducing a modified block copolymer construct in this manner, release of a therapeutic agent can be controlled by selective application of a stimulus.

In an effort to improve the target area of a modified block copolymer construct to a treatment site, such as treatment site 320, the injury site may be isolated prior to delivery. FIG. 3 shows an embodiment of a catheter assembly having two balloons where one balloon is located proximal to treatment site 3 and a second balloon is located distal to treatment site 320. Catheter assembly 300 includes proximal portion 310 and distal portion 315. FIG. 3 shows catheter assembly 300 disposed within blood vessel 300. Catheter assembly 300 has a tandem balloon configuration including proximal balloon 320 and distal balloon 325 aligned in series at a distal portion of the catheter assembly. Catheter assembly 300 also includes primary cannula 330 having a length that extends from a proximal end of catheter assembly 300 (e.g., located external to a patient during a procedure) to connect with a proximal end or skirt of balloon 320. Primary cannula 330 has a lumen therethrough that includes inflation cannula 335 and inflation cannula 340. Inflation cannula 335 extends from a proximal end of catheter assembly 300 to a point within balloon 320. Inflation cannula 335 has a lumen therethrough allowing balloon 320 to be inflated through inflation cannula 335. In this embodiment, balloon 320 is inflated through an inflation lumen separate from the inflation lumen that inflates balloon 325. Inflation cannula 340 has a lumen therethrough allowing fluid to be introduced in the balloon 325 to inflate the balloon. In this manner, balloon 320 and balloon 325 may be separately inflated. Each of inflation cannula 335 and inflation cannula 340 extends from, in one embodiment, the proximal end of catheter assembly 300 through a point within balloon 320 and balloon 325, respectively.

Catheter assembly 300 also includes guidewire cannula 345 extending, in this embodiment, through each of balloon 320 and balloon 325 through a distal end of catheter assembly. Guidewire cannula 345 has a lumen therethrough sized to accommodate a guidewire. In this embodiment, no guidewire is shown within guidewire cannula 345. Catheter assembly 300 may be an over the wire (OTW) configuration or a rapid exchange (RX) type catheter assembly. FIG. 3 illustrates an RX type catheter assembly.

Catheter assembly 300 also includes delivery cannula 350. In this embodiment, delivery cannula extends from a proximal end of catheter assembly 300 through a location between balloon 320 and balloon 325. Secondary cannula 355 extends between balloon 320 and balloon 325. A proximal portion or skirt of balloon 320 connects to a distal end of secondary cannula 355. A distal end or skirt of balloon 320 is connected to a proximal end of secondary cannula 355. Delivery cannula 350 terminates at opening 360 through secondary cannula 355. In this manner, a free treatment agent or a treatment agent encapsulated, suspended, disposed within or loaded into a biodegradable carrier may be introduced between balloon 320 and balloon 325 positioned between treatment site 310.

FIG. 3 shows balloon 320 and balloon 325 each inflated to occlude a lumen of blood vessel 300 and isolate treatment site 320. In one embodiment, each of balloon 320 and balloon 325 are inflated to a point sufficient to occlude blood vessel 300 prior to the introduction of a modified block copolymer construct. A modified block copolymer construct is then introduced.

In the above embodiment, separate balloons having separate inflation lumens are described. It is appreciated, however, that a single inflation lumen may be used to inflate each of balloon 320 and balloon 325. Alternatively, in another embodiment, balloon 325 may be a guidewire balloon configuration such as a PERCUSURG™ catheter assembly where catheter assembly 200 including only balloon 320 is inserted over a guidewire including balloon 325.

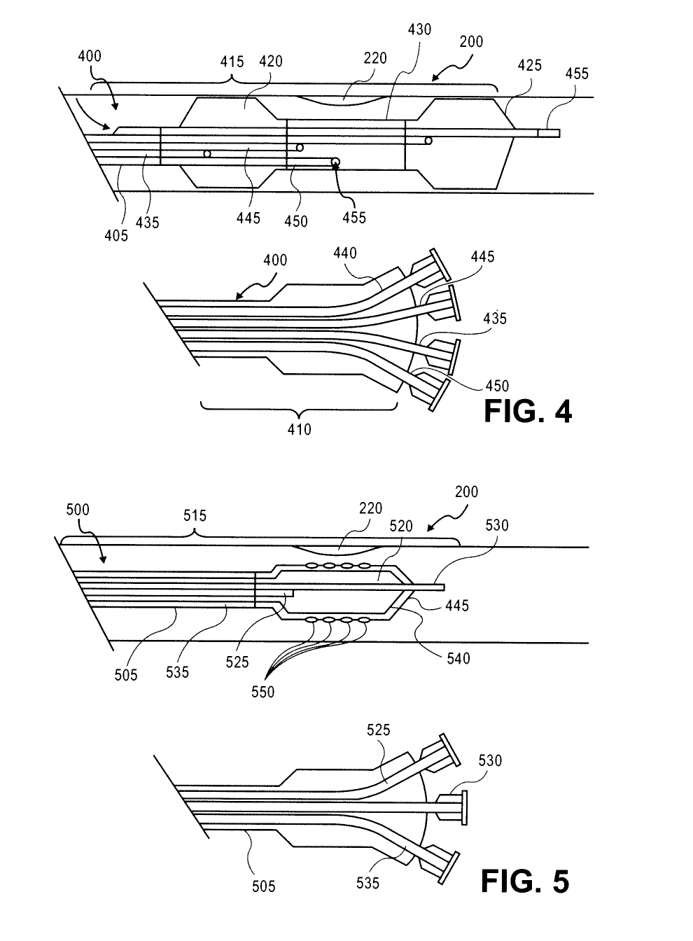

FIG. 4 shows catheter assembly 400 disposed within a lumen of blood vessel 100. Catheter assembly 400 includes proximal portion 410 and distal portion 415. Catheter assembly 400 has a tandem balloon configuration similar to the configuration described with respect to catheter assembly 300 of FIG. 3. In this case, the portion between the tandem balloons is also inflatable. FIG. 4 shows catheter assembly 400 including primary cannula or tubular member 405. In one embodiment, primary cannula 305 extends from a proximal end of the catheter assembly (proximal portion 410) intended to be external to a patient during a procedure, to a point proximal to a region of interest or treatment site within a patient, in this case, proximal to treatment site 220. Representatively, catheter assembly 400 may be percutaneously inserted via femoral artery or a radial artery and advanced into a coronary artery.

Primary cannula 405 is connected in one embodiment to a proximal end (proximal skirt) of balloon 420. A distal end (distal skirt) of balloon 420 is connected to secondary cannula 430. Secondary cannula 430 has a length dimension, in one embodiment, suitable to extend from a distal end of a balloon located proximal to a treatment site beyond a treatment site. In this embodiment, secondary cannula 430 has a property such that it may be inflated to a greater than outside diameter than its outside diameter when it is introduced (in other words, secondary cannula 430 is made of an expandable material). A distal end of secondary cannula 430 is connected to a proximal end (proximal skirt of balloon 425). In one embodiment, each of balloon 420, balloon 425, and secondary cannula 430 are inflatable. Thus, in one embodiment, each of balloon 420, balloon 425, and secondary cannula 430 are inflated with a separate inflation cannula. FIG. 4 shows catheter assembly having inflation cannula 435 extending from a proximal end of catheter assembly 400 to a point within balloon 420; inflation cannula 440 extending from a proximal end of catheter assembly 400 to a point within balloon 425; and inflation cannula 445 extending from a proximal end of catheter assembly 400 to a point within secondary cannula 430. In another embodiment, the catheter assembly may have a balloon configured in a dog-bone arrangement such that inflation of the balloon through a single inflation lumen inflates each of what are described in the figures as separated balloon 420, balloon 425 and secondary cannula 430. Catheter assembly 400 also includes guidewire cannula (no guidewire shown in this embodiment).

By using an expandable structure such as secondary cannula 430 adjacent a treatment site, the expandable structure may be expanded to a point such that a modified block copolymer construct may be dispensed very near or at the treatment site. FIG. 4 shows catheter assembly 400 including delivery cannula 450 extending from a proximal end of catheter assembly 400 through primary cannula 405, through balloon 420 and into secondary cannula 425. Delivery cannula 450 terminates at dispensing port 455 within secondary cannula 430. As viewed, secondary cannula 430 is expandable to an outside diameter less than an expanded outside diameter of balloon 420 or balloon 425 (e.g., secondary cannula 430 has an inflated diameter less than an inner diameter of blood vessel 200 at a treatment site).

FIG. 5 shows another embodiment of a catheter assembly. Catheter assembly 500, in this embodiment, includes a porous balloon through which a treatment agent, such as a modified block copolymer construct, may be introduced. FIG. 5 shows catheter assembly 500 disposed within blood vessel 200. Catheter assembly 500 includes proximal portion 510 and distal portion 515. Catheter assembly 500 has a porous balloon configuration positioned at treatment site 220. Catheter assembly 500 includes primary cannula 505 having a length that extends from a proximal end of catheter assembly 500 (e.g., located external to a patient during a procedure) to connect with a proximal end or skirt of balloon 520. Primary cannula 505 has a lumen therethrough that includes inflation cannula 525. Inflation cannula 525 extends from a proximal end of catheter assembly 500 to a point within balloon 520. Inflation cannula 525 has a lumen therethrough allowing balloon 420 to be inflated through inflation cannula 525.

Catheter assembly 500 also includes guidewire cannula 530 extending, in this embodiment, through balloon 520. Guidewire cannula 530 has a lumen therethrough sized to accommodate a guidewire. In this embodiment, no guidewire is shown within guidewire cannula 530. Catheter assembly 500 may be an over-the-wire (OTW) configuration or rapid exchange (RX) type catheter assembly. FIG. 5 illustrates an OTW type catheter assembly.

Catheter assembly 500 also includes delivery cannula 535. In this embodiment, delivery cannula 535 extends from a proximal end of catheter assembly 500 to proximal end or skirt of balloon 520. Balloon 520 is a double layer balloon. Balloon 520 includes inner layer 540 that is a non-porous material, such as PEBAX, Nylon or PET. Balloon 520 also includes outer layer 545. Outer layer 545 is a porous material, such as extended polytetrafluoroethylene (ePTFE). In one embodiment, delivery cannula 535 is in fluid communication with the space between inner layer 540 and outer layer 545 so that a free treatment agent or a treatment agent encapsulated, suspended, disposed within or loaded into a biodegradable carrier can be introduced between the layers and permeate through pores 550 on outer layer 545 into a lumen of blood vessel 200.

As illustrated in FIG. 5, in one embodiment, catheter assembly is inserted into blood vessel 100 so that balloon 520 is aligned with treatment site 220. Following alignment of balloon 520 of catheter assembly 500, balloon 520 may be inflated by introducing an inflation medium (e.g., liquid through inflation cannula 525). In one embodiment, balloon 520 is only partially inflated or has an inflated diameter less than an inner diameter of blood vessel 200 at treatment site 220. In this manner, balloon 520 does not contact or only minimally contacts the blood vessel wall. A suitable expanded diameter of balloon 520 is on the order of 2.0 to 5.0 mm for coronary vessels. It is appreciated that the expanded diameter may be different for peripheral vasculature. Following the expansion of balloon 520, a treatment agent, such as a modified block copolymer construct, is introduced into delivery cannula 535. The treatment agent can flow through delivery cannula 535 into a volume between inner layer 540 and outer layer 545 of balloon 520. At a relatively low pressure (e.g., on the order of two to four atmospheres (atm)), the treatment agent then permeates through the pores 550 of outer layer 545 into blood vessel 200.

In addition to the device configurations above, devices may have a perfusion lumen incorporated into their respective design, allowing blood flow to bypass the treatment region during the time of treatment.

In the embodiments described with reference to FIGS. 3-5, delivery devices are described for delivering a modified block copolymer construct intra-vascularly (e.g., within a lumen of an artery). In another embodiment, a percutaneously inserted, transluminal delivery device may be selected to deliver a modified block copolymer into the tissue of a blood vessel or extra-luminally (e.g., to a periadventitial space or beyond). Suitable devices are described in commonly-owned U.S. Pat. No. 6,855,124 in which one or more needle cannulas are attached to a proximal taper wall of a balloon and a needle or needles may be advanced into the tissue of a blood vessel or beyond once the balloon is inflated to deliver the modified block copolymer.

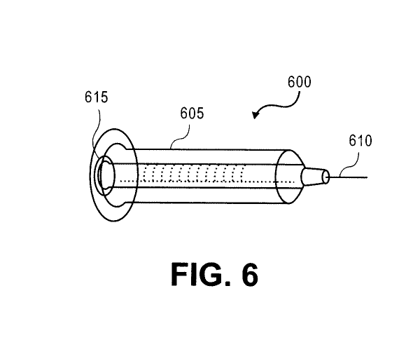

FIG. 6 illustrates an embodiment of a syringe which may be used pursuant to embodiments of the present invention. Syringe 600 may include a body 605, a needle 610 and a plunger 615. A shaft of plunger 615 has an exterior diameter slightly less than an interior diameter of body 605 so that plunger 615 can, in one position, retain a substance in body 605 and, in another position, push a substance through needle 610. Syringes are known by those skilled in the art. In some applications, syringe 600 may be applied directly to a treatment site during an open-chest surgery procedure to deliver a modified block copolymer according to embodiments of the present invention to a treatment site.

From the foregoing detailed description, it will be evident that there are a number of changes, adaptations and modifications of the present invention which come within the province of those skilled in the part. The scope of the invention includes any combination of the elements from the different species and embodiments disclosed herein, as well as subassemblies, assemblies and methods thereof. However, it is intended that all such variations not departing from the spirit of the invention be considered as within the scope thereof.

What is claimed is:

1. A composition comprising:
a carrier comprising a block co-polymer adapted to destabilize upon receiving a stimulus at a treatment site; and
at least one therapeutic agent disposed within the carrier.

2. The composition of claim 1 wherein the stimulus is physical or chemical.

3. The composition of claim 1 wherein the stimulus is internal or external to the treatment site.

4. The composition of claim 2 wherein the stimulus is a physical stimulus comprising one of temperature, electrical field, pressure, sound or radiation.

5. The composition of claim 2 wherein the stimulus is a chemical stimulus comprising a change in one of pH environment or ionic environment at the treatment site.

6. The composition of claim 1 wherein the stimulus is an enzyme.

7. The composition of claim 1 wherein the copolymer is one of polylactide-poly(phenylene oxide), poly(allyl amine hydrochloride)-poly(acrylic acid), poly(dimethylaminoethyl methacrylate)-poly(methyl methacrylate), poly(acrylamide)poly(methyl methacrylate), poly(ethylene glycol)-poly(methyl methacrylate), poly(ethylene glycol)-poly(methacrylic acid), poly(ethylene glycol)-polylactide, poly(ethylene glycol)-collagen, poly(ethylene glycol)-chitosan, N-isopropylacrylamide, 2-hydroxyethylmethacrylate, N-isopropylacrylamide-methacrylic acid-co-octadecyl acrylate or ethylene vinyl alcohol hydrogel.

8. The composition of claim 1 wherein the co-polymer comprises at least one hydrophobic portion and at least one hydrophilic portion.

9. The composition of claim 8 wherein at least one the hydrophobic portion or the hydrophilic portion is biodegradable.

10. The composition of claim 8 wherein the hydrophobic portion comprises one of polylactide, polycaprolactone, polyglycolide, poly(butyl acrylate) and parylene.

11. The composition of claim 8 wherein the hydrophilic portion comprises one of poly(ethylene glycol), polyvinyl alcohol), poly(vinyl pyrrolidone), poly(phosphorylcholine), hyaluronic acid, alginate or collagen.

12. The composition of claim 1 wherein the co-polymer includes a labile bond comprising one of a disulfide, an ortho-ester, an anhydride or a thio ester.

13. The composition of claim 1 wherein the copolymer is modified with at least one chemical moiety adapted to cleave upon receiving a stimulus.

14. The composition of claim 13 wherein the chemical moiety is one of triazene, diazosulfide,

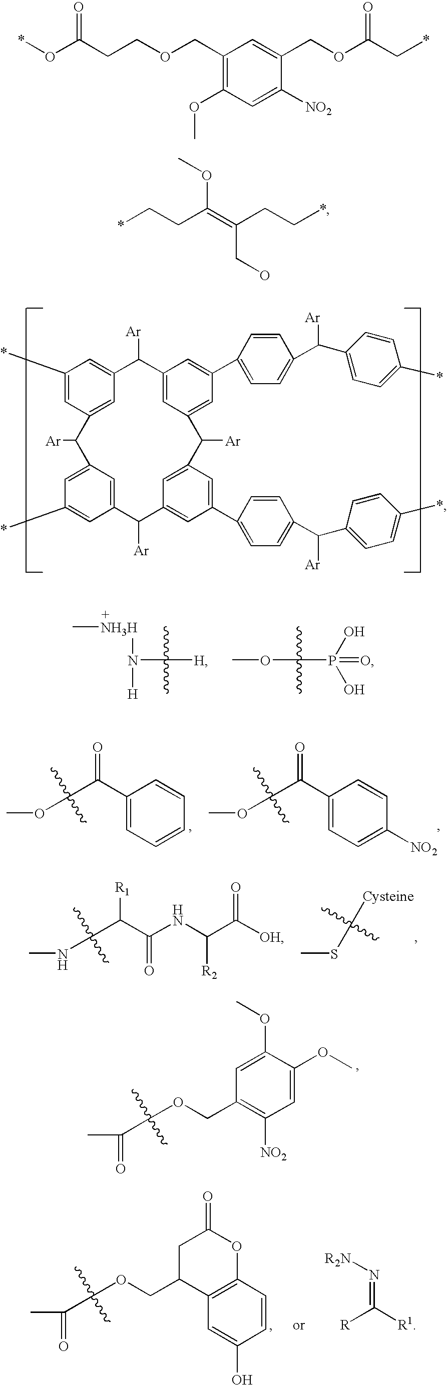

15. The composition of claim 1 wherein the therapeutic agent is one of an anti-inflammatory, an anti-proliferative, an anti-fibrotic, a corticosteroid, a bisphosphonate or Apo-1 mimetic peptide.

16. The composition of claim 6 wherein the enzyme is one of phosphatase, protease or reductase.

17. The composition of claim 1 wherein the carrier is one of a hydrogel, a micelle, a polymerosome, a particle or a coating.

18. The composition of claim 1 wherein the block copolymer is a branched polymer.

19. The composition of claim 1 wherein the hydrophobic portion of the block copolymer is branched.

20. The composition of the claim 19 wherein the branches differ in molecular weight.

21. A composition of claim 19 wherein the branches differ in molecular composition.

22. A composition comprising:
a carrier comprising a block copolymer modified with a chemical moiety wherein the chemical moiety is adapted to destabilize upon receiving a stimulus at a treatment site; and
at least one therapeutic agent disposed within the carrier.

23. The composition of claim 22 wherein the copolymer comprises at least one hydrophobic portion and at least one hydrophilic portion.

24. The composition of claim 23 wherein at least one of the hydrophobic portion or the hydrophilic portion is biodegradable.

25. The composition of claim 23 wherein the hydrophobic portion comprises one of polylactide, polycaprolactone, polyglycolide, poly(butyl acrylate) and parylene

26. The composition of claim 23 wherein the hydrophilic portion comprises one of poly(ethylene glycol), polyvinyl alcohol), poly(vinyl pyrrolidone), poly(phosphorylcholine), hyaluronic acid, alginate or collagen.

27. The composition of claim 23 further comprising a transition region between the hydrophobic portion and the hydrophilic portion.

28. The composition of claim 27 wherein the transition region is one of a disulfide, an ortho-ester, an anhydride or a thio ester.

29. The composition of claim 23 wherein the copolymer is modified with at least one chemical moiety adapted to cleave upon receiving a stimulus.

30. The composition of claim 29 wherein the chemical moiety is any one of claim 14.

31. The composition of claim 22 wherein the stimulus is physical or chemical.

32. The composition of claim 22 wherein the stimulus is internal or external to the treatment site.

33. The composition of claim 31 wherein the stimulus is a physical stimulus comprising one of temperature, electrical field, pressure, sound or radiation.

34. The composition of claim 31 wherein the stimulus is a chemical stimulus comprising a change in one of pH environment or ionic environment at the treatment site.

35. The composition of claim 22 wherein the stimulus is an enzyme.

36. The composition of claim 22 wherein the copolymer is one of polylactide-poly(phenylene oxide), poly(allyl amine hydrochloride)-poly(acrylic acid), poly(dimethylaminoethyl methacrylate)-poly(methyl methacrylate), poly(acrylamide)-poly(methyl methacrylate), poly(ethylene glycol)-poly(methyl methacrylate), poly(ethylene glycol)-poly(methacrylic acid), poly(ethylene glycol)-polylactide, poly(ethylene glycol)-collagen, poly(ethylene glycol)-chitosan, N-isopropylacrylamide, 2-hydroxyethylmethacrylate, N-isopropylacrylamide-methacrylic acid-co-octadecyl acrylate or ethylene vinyl alcohol hydrogel.

37. The composition of claim 22 wherein the therapeutic agent is one of an anti-inflammatory, an anti-proliferative, an anti-fibrotic, a corticosteroid, a bisphosphonate or Apo-1 mimetic peptide.

38. The composition of claim 35 wherein the enzyme is one of phosphatase, protease or reductase.

39. The composition of claim 22 wherein the carrier is one of a hydrogel, a micelle, a polymerosome, a particle or a coating.

40. The composition of claim 22 wherein the block copolymer is a branched polymer.

41. The composition of claim 22 wherein the hydrophobic portion of the block copolymer is branched.

42. The composition of the claim 41 wherein the branches differ in molecular weight.

43. The composition of claim 41 wherein the branches differ in molecular composition.

44. A method of treatment comprising:
inserting a delivery device into a blood vessel of a patient; and
delivering a solution through the delivery device, the solution comprising a carrier comprising a block copolymer modified with a chemical moiety wherein the chemical moiety is adapted to destabilize upon receiving a stimulus at a treatment site and wherein a therapeutic agent is encapsulated, suspended, disposed within or loaded into the carrier.

45. The method of claim 44 wherein a delivery device used to deliver the carrier is one of an infusion catheter, a porous balloon catheter, a needle injection catheter, a double balloon catheter or a syringe.

46. The method of claim 44 wherein the copolymer comprises a hydrophobic portion and a hydrophilic portion.

47. The method of claim 46 further comprising a transition region between the hydrophobic portion and the hydrophilic portion.

48. The method of claim 47 wherein the transition region is one of a disulfide, an ortho-ester, an anhydride or a thio ester.

49. The method of claim 44 wherein the copolymer is modified with at least one chemical moiety adapted to cleave upon receiving a stimulus.

50. The method of claim 49 wherein the chemical moiety is any one of claim 14.

51. The method of claim 44 wherein the stimulus is physical or chemical.

52. The method of claim 44 wherein the stimulus is internal or external to the treatment site.

53. The method of claim 44 wherein the stimulus is supplied from outside the patient.

54. The method of claim 44 wherein the stimulus is localized to the site of treatment.

55. The method of claim 54 wherein the stimulus is provided by means of a catheter.

56. The method of claim 51 wherein the stimulus is a physical stimulus comprising one of temperature, electrical field, pressure, sound or radiation.

57. The method of claim 51 wherein the stimulus is a chemical stimulus comprising a change in one of pH environment or ionic environment at the treatment site.

58. The method of claim 44 wherein the stimulus is an enzyme.

59. The method of claim 44 wherein the copolymer is one of polylactide-poly(phenylene oxide), poly(allyl amine hydrochloride)-poly(acrylic acid), poly(dimethylaminoethyl methacrylate)-poly(methyl methacrylate), poly(acrylamide)-poly(methyl methacrylate), poly(ethylene glycol)-poly(methyl methacrylate), poly(ethylene glycol)-poly(methacrylic acid), poly(ethylene glycol)-polylactide, poly(ethylene glycol)-collagen, poly(ethylene glycol)-chitosan, N-isopropylacrylamide, 2-hydroxyethylmethacrylate, N-isopropylacrylamide-methacrylic acid-co-octadecyl acrylate or ethylene vinyl alcohol hydrogel.

60. The method of claim 44 wherein the therapeutic agent is one of an anti-inflammatory, an anti-proliferative, an anti-fibrotic, a corticosteroid, a bisphosphonate or Apo-1 mimetic peptide.

61. The method of claim 58 wherein the enzyme is one of phosphatase, protease or reductase.

62. The method of claim 44 wherein the carrier is one of a hydrogel, a micelle, a polymerosome, a particle or a coating.
